(19) United States
(12) Patent Application Publication
(10) Pub. No.: US 2010/0130415 A1
(43) Pub. Date: May 27, 2010

(54) Stem cell secreted product derived compositions for wound treatment

(76) Inventors: Michael Cohen, West Orange, NJ (US); Jacob Cohen, West Orange, NJ (US)

Correspondence Address: HOWREY LLP-HN, C/O IP DOCKETING DEPARTMENT, 2941 FAIRVIEW PARK DRIVE, SUITE 200, FALLS CHURCH, VA, 22042-7195, US

(21) Appl. No.: 12/448,436

(22) PCT Filed: Dec. 19, 2007
(86) PCT No.: PCT/US07/25836

§ 371 Date: Feb. 2, 2010

Related U.S. Application Data
(60) Provisional application No. 60/875,560, filed on Dec. 19, 2006.

(51) Int. Cl. A61K 38/17; A61P 17/02

(52) U.S. Cl. 514/12

(57) ABSTRACT

Compositions including formulations comprising secreted products obtained from the culture medium of stem cells, such as umbilical cord blood stem cells, or embryonic germ cell derivatives, or embryonic stem cells, are provided for enhancement of wound healing. Further compositions contain components identified in such culture medium to enhance wound healing. Methods for using the compositions and formulations for enhancing wound healing are also provided. Wounds to both soft and bony tissues are encompassed, and include wounds created by surgical procedures.

### BACKGROUND

Wounds are internal or external bodily injuries or lesions caused by physical means, such as mechanical, chemical, viral, bacterial, fungal and other pathogenic organisms, or thermal means, which disrupt the normal continuity of tissue structure. Such bodily injuries include contusions, wounds in which the skin is unbroken, incisions, wounds in which the skin is broken cutting instrument, and lacerations, wounds in which the skin is broken by a dull or, blunt instrument. Wounds may be caused by accident, surgery, pathological organisms, or by surgical procedures.

People afflicted with long-term illness run the risk of getting bed sores, pressure sores and a myriad of skin irritations and chronic wounds. Cancer patients, in particular breast cancer patients, treated with radiation face the risk of skin burns. Wound healing after surgical intervention has been historically problematic. Healing of skin grafts and of plastic surgery procedures are susceptible to poor or slow healing. The benefits of surgery, even in life threatening situations, are offset by the formation of disfiguring scar tissue. Adult wound healing is characterized by fibrosis, scarring, and sometimes by contracture.

Wound healing consists of a series of processes whereby injured tissue is repaired, specialized tissue is regenerated, and new tissue is reorganized. Wound healing consists of three major phases: a) an inflammation stage (0-3 days), b) proliferation stage (3-12 days), and c) a remodeling phase (3 days to 6 months). During the inflammation phase, platelet aggregation and clotting from a matrix which traps the plasma proteins and blood cells to induce the influx of various types of cells. During the cellular proliferation phase, new connective or granulation tissue and blood vessels are formed. During the remodeling phase, granulation tissue is replaced by a network of collagen and elastin fibers leading to the formation of scar tissue.

A problematic wound does not follow the normal time table for the healing process as described above. A problematic wound could fail to follow the normal healing process for any number of reasons, including nutrition, vascular status, metabolic factors, age, immune status, drug therapy, neurologic status and psychologic status, among others. Several local factors also play an important role in wound healing, including the presence of necrotic tissue in the area, infection, foreign body presence, degree of desiccation, presence of edema, pressure, friction, shear maceration and dermatitis.

Methods and compositions for increasing the healing rate and strength of healed wounds, whether external or internal to the body, accidental, pathologic or iatrogenic, and including burns, would be a welcome addition to medical practice.

### SUMMARY

In one embodiment, a composition is provided for the enhancement of wound healing, the composition comprising secreted products from embryonic germ cell derivatives in a pharmaceutically-acceptable carrier. In another embodiment, the embryonic germ cell derivatives are human embryoid body-derived cells. In another embodiment, the human embryoid body derived cells are LVEC cells or SDEC cells. In other embodiments, the embryonic germ (EG) derivatives are from mouse, pig, chicken, or human. In another embodiment, enhancement of wound healing comprises accelerated rate of epithelial regeneration. In another embodiment, enhancement of wound healing comprises increased wound tensile strength.

In another embodiment, a composition is provided for the enhancement of wound healing, the composition comprising secreted products from stem cells in a pharmaceutically-acceptable carrier. In one embodiment the stem cells are embryonic stem (ES) cells. In one embodiment, the stem cells are umbilical cord stem cells. In another embodiment, the umbilical cord stem cells are USCC cells. In another embodiment, enhancement of wound healing comprises accelerated rate of epithelial regeneration. In another embodiment, enhancement of wound healing comprises increased wound tensile strength.

In another embodiment, a method for enhancing wound healing is provided comprising applying to a wound site a composition comprising secreted products from embryonic germ cell derivatives in a pharmaceutically-acceptable carrier. In another embodiment, the embryonic germ cell derivatives are human embryoid body-derived cells. In another embodiment, the human embryoid body derived cells are LVEC cells or SDEC cells. In other embodiments, the embryonic germ (EG) derivatives are from mouse, pig, chicken, or human. In another embodiment, enhancement of wound healing comprises accelerated rate of epithelial regeneration. In another embodiment, enhancement of wound healing comprises increased wound tensile strength.

In another embodiment, a method for enhancing wound healing is provided comprising applying to a wound site a composition comprising secreted products from stem cells in a pharmaceutically-acceptable carrier. In one embodiment the stem cells are embryonic stem cells. In one embodiment, the stem cells are umbilical cord stem cells. In another embodiment, the umbilical cord stem cells are USCC cells. In another embodiment, enhancement of wound healing comprises accelerated rate of epithelial regeneration. In another embodiment, enhancement of wound healing comprises increased wound tensile strength.

In another embodiment, a composition for enhancing wound healing comprises the following components or homologues or analogues thereof: elastase 2A; prostaglandin I2; prostaglandin E2; adam metallopeptidase with thrombospondin type 1 motif 5; bone morphogenetic protein 1; bone morphogenetic protein 6; chemokine (C-C motif) ligand 2; chemokine (C-C motif) ligand 20; chemokine (C-X-C motif) ligand 1; chemokine (C-X-C motif) ligand 2; chemokine (C-X-C motif) ligand 3; chemokine (C-X-C motif) ligand 5; chemokine (C-X-C motif) ligand 6; chemokine (C-X-C motif) ligand 9; colony stimulating factor 2; colony stimulating factor 3; gremlin 1, cysteine knot superfamily, homolog (Xenopus laevis); gremlin 2, cysteine knot superfamily, homolog (Xenopus laevis); heparin-binding EGF-like growth factor; natriuretic peptide precursor B; pleiotrophin; pre-B-cell colony enhancing factor 1; tumor necrosis factor (ligand) superfamily, member 4; and tumor necrosis factor receptor superfamily, member 11b. In another embodiment, methods are provided for treating wounds or enhancing wound healing by applying to a wound the aforementioned composition, optionally in a carrier.

In another embodiment, a composition is provided for enhancing wound healing comprising the following components or homologues or analogues thereof: elastase 2A; prostaglandin I2; prostaglandin E2; amphiregulin; fibroblast growth factor 2; fibroblast growth factor 7; G protein-coupled receptor, family C, group 5, member B; and GABA(a) receptor-associated protein like 1. In another embodiment, methods are provided for treating wounds or enhancing wound healing by applying to a wound the aforementioned composition, optionally in a carrier.

The subject matter regarded as the invention is particularly pointed out and distinctly claimed in the concluding portion of the specification. The invention, however, both as to organization and method of operation, together with objects, features, and advantages thereof, may best be understood by reference to the following detailed description.

### DETAILED DESCRIPTION OF THE EMBODIMENTS

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

This application provides in one embodiment secreted products from cultures of embryonic germ (EG) cell derivatives, and in another embodiment secreted products from stem cells, that offer benefit in addressing wound healing.

As noted above, wound healing consists of a series of processes whereby injured tissue is repaired, specialized tissue is regenerated, and new tissue is reorganized. Wound healing consists of three major phases: a) an inflammation stage (0-3 days), b) proliferation stage (3-12 days), and c) a remodeling phase (3 days to 6 months). During the inflammation phase, platelet aggregation and clotting from a matrix which traps the plasma proteins and blood cells to induce the influx of various types of cells. During the cellular proliferation phase, new connective or granulation tissue and blood vessels are formed. During the remodeling phase, granulation tissue is replaced by a network of collagen and elastin fibers leading to the formation of scar tissue. The compositions and methods of the invention enhance wound healing typically during at least one of the aforementioned phases, or at two of the three phases, or during all three phases.

Selections of components in the wound healing compositions embodied herein as well as the methods are described in more detail below. These embodiments are merely exemplary and non-limiting.

EG cell derivatives. In the practice of certain of the embodiments herein, human embryonic germ (EG) cell derivatives are used as a source of the secreted products for the wound healing compositions and methods described herein. EG cells can be generated and cultured essentially as described in U.S. Pat. No. 6,090,622. The starting material for isolating cultured embryonic germ (EG) cells is tissues and organs comprising primordial germ cells (PGCs). For example, PGCs may be isolated over a period of about 3 to 13 weeks post-fertilization (e.g., about 9 weeks to about 11 weeks from the last menstrual period) from embryonic yolk sac, mesenteries, gonadal anlagen, or genital ridges from a human embryo or fetus. Alternatively, gonocytes of later testicular stages can also provide PGCs. In one embodiment, the PGCs are cultured on mitotically inactivated fibroblast cells (e.g., STO cells) under conditions effective to derive EGs. The resulting human EG cells resemble murine ES or EG cells in morphology and in biochemical histotype. The resulting human EG cells can be passaged and maintained for at least several months in culture.

Embryoid body-derived cells. In the practice of certain of the embodiments described herein, typically embryoid body-derived cells, derived from embryonic germ cells as mentioned above, are used to provide secreted products. Methods for preparing embryoid body-derived cells are described in U.S. Patent Application Publication No. 2003/0175954, published Sep. 18, 2003, and based on Ser. No. 09/767,421, and incorporated herein by reference in its entirety. Such cells can be derived from human embryoid bodies (EBs), which are in turn produced by culturing EG cells, as described above. Methods for making EBs are described below. Unlike EBs, which are large, multicellular three-dimensional structures, embryoid body-derived cells grow as a monolayer and can be continuously passaged. Although EBD cells are not immortal, they display long-term growth and proliferation in culture. Mixed cell EBD cultures and clonally isolated EBD cell lines simultaneously express a wide array of mRNA and protein markers that are normally associated with cells of multiple distinct developmental lineages, including neural (ectodermal), vascular/hematopoietic (mesodermal), muscle (mesodermal) and endoderm lineages. Mesodermal cells include, for example, connective tissue cells (e.g., fibroblasts) bone, cartilage (e.g., chondrocytes), muscle (e.g., myocytes), blood and blood vessels, lymphatic and lymphoid organs cells, neuronal cells, pleura, pericardium, kidney, gonad and peritoneum. Ectodermal cells include, for example, epidermal cells such as those of the nail, hair, glands of the skin, nervous system, the external organs (e.g., eyes and ears) and the mucosal membranes (e.g., mouth, nose, anus, vaginal). Endodermal cells include, e.g., those of the pharynx, respiratory tract, digestive tract, bladder, liver, pancreas and urethra cells. The growth and expression characteristics of EBD cells reveal an uncommitted precursor or progenitor cells phenotype.

Human embryoid bodies (EBs) form spontaneously in human primordial germ cell-derived stem cell cultures that have been maintained in the presence of leukemia inhibitory factor (LIF) (e.g., human recombinant leukemia inhibitory factor) at about, e.g., 1000 units/ml, basic fibroblast growth factor (bFGF), at about 1 ng/ml, and forskolin at about 10 μM for greater than about one month, and, in some situations, as long as three to six months. EBs are also formed when these factors are withdrawn. Additional factors can be added to enhance or direct this process, including, but not limited to, retinoic acid, dimethylsulfoxide (DMSO), cAMP elevators such as forskolin, isobutylmethylxanthine, and dibutryl cAMP, cytokines such as basic fibroblast growth factor, epidermal growth factor, platelet derived growth factor (PDGF and PDGF-AA) nerve growth factor, T3, sonic hedgehog (Shh or N-Terminal fragment), ciliary neurotrophic factor (CNTF), erythropoietin (EPO) and bone morphogenic factors. The foregoing list is merely exemplary and not intended at be limiting.

Moreover, and as will be discussed further below, embryoid body-derived cells used in the practice of the embodiments herein include cells as described above as well as those that can be transformed or infected. Guidance for methods of so doing may be found in U.S. Patent Application Publication 200310175954. Genetic manipulation for the purposes described herein include those that increase the secretion of products beneficial for the treatment of skin and its various aspects as described above.

By way of non-limiting example as to the preparation of EG cell derivatives, EBs are physically removed from the stem cell culture medium where they are formed (see above), and placed in a calcium and magnesium-free phosphate-buffered saline (PBS). The EBs are then sorted into categories by gross morphology, e.g., cystic or solid. After sorting, the EBs are transferred to a mixture of one mg/ml collagenase and dispase enzyme (Boehringer Mannheim), and incubated for 30 minutes to three hours at 37 C.; during this time they are manually agitated or triturated every about 10 to 30 minutes. Other dissociation treatments can be used, e.g., the individual or combined use of several different types of collagenase, dispase I, dispase II, hyaluronidase, papain, proteinase K, neuraminidase and/or trypsin. Each treatment requires optimization of incubation length and effectiveness; cell viability can be monitored visually or by trypan blue exclusion followed by microscopic examination of a small aliquot of the disaggregation reaction. One collagenase/dispase disaggregation protocol calls for incubation for about 30 minutes at 37 C.; this results in between about 10% and 95% of the EB constituent cells disaggregated into single cells. Large clumps of cell may remain intact.

After disaggregation, one to five mls of growth medium are added to the cells. One exemplary medium comprises EGM2-MV medium (Clonetics/Cambrex) with about 10 to 20% fetal calf serum supplemented with antibiotics, e.g., penicillin and streptomycin. The cell suspension is then centrifuged at about 100 to 500 g for about five minutes. The supernatant is then removed and replaced with fresh growth media. The cells are resuspended and plated into a tissue culture vessel that can be coated with cells or typically a biomatrix. In a typical embodiment, collagen type I is used as the substrate.

EBD cells obtained from 4 to 8 EBs can be resuspended in media, e.g., about three ml media (e.g., RPMI), and plated (e.g., into a 3.5 cm diameter plate) onto a surface that has been coated with a collagen (e.g., human type I collagen). The culture media is replaced every two to three days. This is a general method that will allow a wide variety of cell types to proliferate.

In one embodiment EBDs are utilized to produce secreted products for the applications herein. As described above, EBD cells can be clonally isolated and are capable of robust and long-term proliferation in culture. EBD cells are grown and maintained in culture medium or growth medium. Examples of suitable culture media include EGM2-MV medium as mentioned above, knockout DMEM (from GibcoBRL, Life Technologies), Hepatostim (BD Biosciences) and DMEM medium containing knockout serum (Invitrogen) or plasminate, to name only a few examples.

LVEC and SDEC cells. In one embodiment, secreted products from LVEC cells are used in the aforementioned compositions and methods for treating wounds and enhancing wound healing. In another embodiment, secreted products from SDEC cells are used in the aforementioned compositions. In yet another embodiment, secreted products from embryoid body derived cells are used in the aforementioned compositions. In still a further embodiment, secreted products from embryonic germ cells derivatives are used in the aforementioned compositions.

Umbilical cord stem cells. With regard to the embodiments of the invention wherein secreted products from stem cells are used, in the practice of certain of the embodiments herein, human umbilical cord stem cells are used as a source of the secreted products for the wound healing compositions and methods of use herein. They can be grown in accordance with standard protocols, such as, by way of non-limited example, in USSC media (low glucose DMEM with Glutamax, Invitrogen 10567-014), 10⁻⁷ M dexamethasone (Sigma), 100 U/ml penicillin and 0.1 mg/ml streptomycin. In a further example, medium was changed after 48 hrs then every 2-3 days following. On day 14, proliferating cells were passaged 1:3 into new flasks by using 0.25% trypsin/EDTA and neutralized by trypsin neutralization solution. Every 5 to 7 days cells were similarly passaged. The phenotype of passage 5 umbilical cord stem cells was CD31 (2%), CD34 (0%), CD44 (97%), CD50 (0%), CD71 (47%), CD90, (96%), CD106 (0%). The conditioned medium of such cell cultures provides the composition herein for use in wound healing applications.

In another example, conditioned media was prepared by plating umbilical cord stem cells at 1 million cells per 10 cm plate into 12 mls of HuES media. After 24 hrs media was harvested and sterile filtered. The media comprises secreted products useful for the various embodiments described herein.

USSC Cells. In another example, cells useful for the preparation of secreted products can be obtained as described in Koglar G, Sensken S, Airey J A, Trapp T, Muschen M, Feldhahn N, et al. 2004. A new human somatic stem cell from placental cord blood with intrinsic pluripotent differentiation potential. J. Exp. Med. 200, 123-135. A somatic stem cell population termed USSC was grown adherently and expanded to 10¹⁵ without losing developmental potential. In vitro, umbilical cord stem cells showed homogeneous differentiation into hematopoietic and neural cell lineage. Immunoassay of umbilical cord stem cells showed CD34, CD45, CD106 negative and CD44 and CD90 positive cell phenotype. Secreted products obtained therefrom are provided in a wound healing formulation for the purposes described herein.

In other embodiment, CD34 positive umbilical cord stem cells are used to provide the secreted products for the purposes embodied herein.

Embryonic Stern Cells. Other stem cells can be used in the practice of the various embodiments of the invention include embryonic stem cells. Embryonic stem (ES) cells are derived from the inner cell mass of preimplantation embryos. ES cells are pluripotent and are capable of differentiating into cells derived from all three embryonic germ layers. The traditional method used to derive mouse and human embryonic stem (ES) cells involves the use of support cells termed feeder cells or layers. These support cells provide a poorly understood set of signals that promote the conversion from blastocyst inner cell mass (ICM) cells to proliferating ES cells. Most commonly, primary cultures of mouse embryo fibroblasts are used as support cells for both mouse and human ES cultures. The requirement for support cells is not lost following derivation, and ES cell cultures are most commonly maintained on feeder layers until differentiation is desired. WO/9920741 describes the growth of ES cells in a nutrient serum effective to support the growth of primate-derived primordial stem cells and a substrate of feeder cells or an extracellular matrix component derived from feeder cells. The medium further includes non-essential amino acids, an anti-oxidant, and growth factors that are either nucleosides or a pyruvate salt. U.S. Pat. No. 6,642,048 reports growth of ES cells in feeder-free culture, using conditioned medium from such cells. U.S. Pat. No. 6,800,480 describes a cell culture medium for growing primate-derived primordial stem cells comprising a low osmotic pressure, low endotoxin basic medium comprising a nutrient serum and an extracellular matrix derived from the feeder cells. The medium further includes non-essential amino acids, an anti-oxidant (for example, beta-mercaptoethanol), and, optionally, nucleosides and a pyruvate salt.

Secreted Products. Secreted products from either embryonic germ (EG) cell derivatives and stem cells, or other cells described herein, are also referred to as “conditioned medium,” a term that refers to a growth medium that is further supplemented by factors derived from media obtained from cultures of cells, in these cases, embryonic germ (EG) cell derivatives, embryoid body-derived cells or stem cells such as umbilical cord stem cells. The term “effective amount” as used herein is the amount of such described factor as to permit a beneficial effect on wound healing when formulated and applied as described herein.

Components from conditioned medium. In further embodiments, certain combinations of components identified to be present within secreted products from cultures of embryonic germ (EG) cell derivatives or umbilical cord blood stem cells are found to provide beneficial properties for enhancing healing of wounds. Such properties include but are not limited to inducing growth of skin cells and increasing the number of keratinocytes in the skin.

Such compositions comprise, in one embodiment, elastase 2A; prostaglandin I2; prostaglandin E2; adam metallopeptidase with thrombospondin type 1 motif 5; bone morphogenetic protein 1; bone morphogenetic protein 6; chemokine (C-C motif) ligand 2; chemokine (C-C motif) ligand 20; chemokine (C-X-C motif) ligand 1; chemokine (C-X-C motif) ligand 2; chemokine (C-X-C motif) ligand 3; chemokine (C-X-C motif) ligand 5; chemokine (C-X-C motif) ligand 6; chemokine (C-X-C motif) ligand 9; colony stimulating factor 2; colony stimulating factor 3; gremlin 1, cysteine knot superfamily, homolog (Xenopus laevis); gremlin 2, cysteine knot superfamily, homolog (Xenopus laevis); heparin-binding EGF-like growth factor; natriuretic peptide precursor B; pleiotrophin; pre-B-cell colony enhancing factor 1; tumor necrosis factor (ligand) superfamily, member 4; and tumor necrosis factor receptor superfamily, member 11b.

Compositions for treatment of wounds may comprise the individual components at a concentration from about 0.000001 to 1.0%. In other embodiments, the components are present in the same relative proportions as they are present in conditioned medium. In a further embodiment the components are present at the same relative proportions as they are present in conditioned medium, but at an overall concentration higher than or lower than present in conditioned medium. As noted herein below, in certain formulations such as a liposome formulation, the aforementioned components may be present at higher amounts within the interior of the liposomes but at a lower concentration in the formulation based on the overall liposome content of the formulation.

The aforementioned components of the compositions embodied herein are described in further detail below. The descriptions that are provided are meant to be merely exemplary of the components and biological activities, are not intending to be limiting. A non-limiting list of synonyms and a brief description of the individual components is provided yet Applicant is not bound to the descriptions thereof. Reference is provided to the Uniprot (Universal Protein Resource) data base (www.pir.uniprot.org), a source of sequence and other information on these components. Exemplary, non-limiting information is provided. Homologues of the components include, in the instance where components are proteins, of homologues of various mammalian species, including but not limited to human, chimpanzee, pig, rat, and mouse, as well as muteins and other functional, variants, analogs, and modifications of the proteins that provide the same or similar biological activity. Homologues of the organic compounds herein include analogs, variants, adducts, modifications and the like, with the same or similar biological activity as the compound described. Analogs may have enhanced activity.

Elastase 2A (synonyms: ELA1, Elastase-2A precursor, PE-1) is a pancreatic serine protease that hydrolyzes elastin, a fibrous, insoluble protein of connective tissue. Non-limiting examples of elastase 2A include UniProt entries P08217 Q6ISN8, and Q6ISU5, and homologues thereof.

Prostaglandin I2 (synonyms: PGI2, prostacyclin) or 5-[7-hydroxy-8-(3-hydroxyoct-1-enyl)-4-oxabicyclo[3.3.0]oct-3-ylidene] pentanoic acid, is a member of the prostanoids and is known to prevent platelet formation and clumping involved in blood clotting. It is also an effective vasodilator. Non-limiting analogs include iloprost and cisaprost.

Prostaglandin E2 (synonym: PGE2), or (Z)-7-((1R,2R,3R)-3-hydroxy-2-((S,E)-3-hydroxyoct-1-enyl)-5-oxocyclopentyl)hept-5-enoic acid, is a prostanoid that is released by blood vessel walls in response to infection or inflammation that acts on the brain to induce fever.

Adam metallopeptidase with thrombospondin type 1 motif, 5 (synonyms: ADAMTS11, ADAM-TS 11, ADAM-TS5, ADAM-TS 5, ADAMTS-5 precursor, A disintegrin and metalloproteinase with thrombospondin motifs 5, ADMP2, ADMP-2, aggrecanase-2, FLJ36738) is a member of the ADAMTS (a disintegrin and metalloproteinase with thrombospondin motifs) protein family. The enzyme encoded by this gene contains two C-terminal TS motifs and functions as aggrecanase to cleave aggrecan, a major proteoglycan of cartilage. Non-limiting examples include UniProt entries Q9UNA0, Q52LV4, Q9UKP2, and homologues thereof.

Bone morphogenetic protein 1 (synonyms: BMP-1, bone morphogenetic protein 1 precursor, FLJ44432, mammalian tolloid protein, mild, PCOLC, PCP, procollagen C-proteinase, TLD) is a protein that belongs to the peptidase M12A family of proteins. It induces bone and cartilage development. It is a metalloprotease that cleaves the C-terminus of procollagen I, II and III. It has an astacin-like protease domain. It has been shown to cleave laminin 5 and is localized in the basal epithelial layer of bovine skin. Non-limiting examples include UniProt entries P13497, Q59F71, Q3MIM8, and homologues thereof.

Bone morphogenetic protein 6 (synonyms: BMP-6, bone morphogenetic protein 6 precursor, VGR, VGR1) is a polypeptide that is a member of the TGFβ superfamily of proteins. Bone morphogenetic proteins are known for their ability to induce the growth of bone and cartilage. BMP6 is able to induce all osteogenic markers in mesenchymal stem cells. Non-limiting examples include UniProt entries P22004, Q5TCP3, Q4VBA3, and homologues thereof.

Chemokine (C-C motif) ligand 2 (synonyms: GDCF-2, GDCF-2HC11, HC11, HSMCR30, MCAF, MCP1, MCP-1, MGC9434, monocyte chemoattractant protein 1, monocyte chemotactic and activating factor, monocyte chemotactic protein 1, monocyte secretory protein JE, SCYA2, small inducible cytokine A2 precursor, SMC-CF, monocyte chemotactic protein 1, homologous to mouse Sig-je, small inducible cytokine A2, monocyte chemotactic protein 1, homologous to mouse Sig-je) displays chemotactic activity for monocytes and basophils but not for neutrophils or eosinophils. It has been implicated in the pathogenesis of diseases characterized by monocytic infiltrates, like psoriasis, rheumatoid arthritis and atherosclerosis. It binds to chemokine receptors CCR2 and CCR4. Non-limiting examples include UniProt entries P13500, Q6UZ82, Q9UDF3, and homologues thereof.

Chemokine (C-C motif) ligand 20 (synonyms: Beta chemokine exodus-1, CC chemokine LARC, CKb4, exodus-1, LARC, Liver and activation-regulated chemokine, macrophage inflammatory protein 3 alpha, MIP3A, MIP-3a, MIP-3-alpha, SCYA20, small inducible cytokine A20 precursor, ST38) is a small cytokine belonging to the CC chemokine family. It is strongly chemotactic for lymphocytes and weakly attracts neutrophils. It is implicated in the formation and function of mucosal lymphoid tissues via chemoattraction of lymphocytes and dendritic cells towards the epithelial cells surrounding these tissues. CCL20 elicits its effects on its target cells by binding and activating the chemokine receptor CCR6. Non-limiting examples include UniProt entries P78556, Q53S51, Q99664, and homologues thereof.

Chemokine (C-X-C motif) ligand I (synonyms: GRO, GRO1, GROa, GROA, GRO-alpha(1-73), growth-regulated protein alpha precursor, melanoma growth stimulatory activity, MGSA, MGSA-a, MGSA alpha, NAP-3, neutrophil-activating protein 3, SCYB1) is a small cytokine belonging to the CXC chemokine family that was previously called GRO1 oncogene, Neutrophil-activating protein 3 (NAP-3) and melanoma growth stimulating activity, alpha (MSGA-α). It is secreted by human melanoma cells, has mitogenic properties and is implicated in melanoma pathogenesis. CXCL1 is expressed by macrophages, neutrophils and epithelial cells, and has neutrophil chemoattractant activity. CXCL1 plays a role in spinal cord development by inhibiting the migration of oligodendrocyte precursors and is involved in the processes of angiogenesis, inflammation, wound healing, and tumorigenesis. This chemokine elicits its effects by signaling through the chemokine receptor CXCR2. Non-limited examples include UniProt entries P09341, Q6LD34, and homologues thereof.

Chemokine (C-X-C motif) ligand 2 (synonyms: CINC-2a, GRO2, GROb, GROB, Gro-beta, Growth-regulated protein beta, Macrophage inflammatory protein 2-alpha precursor, MGSA-b, MGSA beta, MIP2, MIP2A, MIP-2a, MIP2-alpha, SCYB2) is a small cytokine belonging to the CXC chemokine family that is also called macrophage inflammatory protein 2-alpha (MIP2-alpha), Growth-regulated protein beta (Gro-beta) and Gro oncogene-2 (Gro-2). CXCL2 is 90% identical in amino acid sequence as a related chemokine, CXCL1. This chemokine is secreted by monocytes and macrophages and is chemotactic for polymorphonuclear leukocytes and hematopoietic stem cells. The gene for CXCL2 is located on human chromosome 4 in a cluster of other CXC chemokines. CXCL2 mobilizes cells by interacting with a cell surface chemokine receptor called CXCR2. Non-limiting examples include UniProt entries P19875, Q6LD33, Q6FGD6, and homologues thereof.

Chemokine (C-X-C motif) ligand 3 (synonyms: CINC-2b, GRO3, GROg, GROG, GRO-gamma, GRO-gamma(1-73), growth-regulated protein gamma, macrophage inflammatory protein 2-beta precursor, MGSA gamma, MIP2B, MIP-2b, MIP2-beta, SCYB3) is a small cytokine belonging to the CXC chemokine family that is also known as GRO3 oncogene (GRO3), GRO protein gamma (GROg) and macrophage inflammatory protein-2-beta (MIP2b). CXCL3 controls migration and adhesion of monocytes and mediates it effects on its target cell by interacting with a cell surface chemokine receptor called CXCR2. Non-limiting examples include UniProt entries P19876, Q6LD32, Q4W5H9, and homologues thereof.

Chemokine (C-X-C motif) ligand 5 (synonyms: ENA78, ENA-78, ENA-78(1-78), epithelial-derived neutrophil-activating protein 78, neutrophil-activating peptide ENA-78, SCYB5, small inducible cytokine B5 precursor) is a small cytokine belonging to the CXC chemokine family that is also known as epithelial-derived neutrophil-activating peptide 78 (ENA-78). It is produced following stimulation of cells with the inflammatory cytokines interleukin-1 or tumor necrosis factor-alpha. Expression of CXCL5 has also been observed in cosinophils, and can be inhibited with the type II interferon IFN-γ. This chemokine stimulates the chemotaxis of neutrophils possessing angiogenic properties. It elicits these effects by interacting with the cell surface chemokine receptor CXCR2. CXCL5 has been implicated in connective tissue remodelling. Non-limiting examples include UniProt entries P42830, Q6I9S7, Q96QE1, and homologues thereof.

Chemokine (C-X-C motif) ligand 6 (synonyms: chemokine alpha 3, CKA-3, GCP2, GCP-2, granulocyte chemotactic protein 2, SCYB6, small inducible cytokine B6 precursor) is a small cytokine belonging to the CXC chemokine family that is also known as granulocyte chemotactic protein 2 (GCP-2). As its former name suggests, CXCL6 is a chemoattractant for neutrophilic granulocytes. It elicits its chemotactic effects by interacting with the chemokine receptors CXCR1 and CXCR2. Non-limiting examples include UniProt entries P80162, Q4W5D4, O00172, and homologues thereof.

Chemokine (C-X-C motif) ligand 9 (synonyms: CMK, crg-10, Gamma interferon-induced monokine, Humig, MIG, SCYB9, Small inducible cytokine B9 precursor) is a small cytokine belonging to the CXC chemokine family that is also known as Monokine induced by gamma interferon (MIG). CXCL9 is a T-cell chemoattractant, which is induced by IFN-γ. CXCL9 elicits its chemotactic function by interacting with the chemokine receptor CXCR3. Non-limiting examples include UniProt entries Q07325, Q503B4, and homologues thereof.

Colony stimulating factor 2 (granulocyte-macrophage) (synonyms: colony-stimulating factor, CSF, GMCSF, GM-CSF, granulocyte-macrophage colony-stimulating factor precursor, MGC131935, molgramostin, sargramostim) is a cytokine that functions as a white blood cell growth factor. GM-CSF stimulates stern cells to produce granulocytes (neutrophils, eosinophils, and basophils) and monocytes. Monocytes exit the circulation and migrate into tissue, whereupon they mature into macrophages. It is thus part of the immune/inflammatory cascade, by which activation of a small number of macrophages can rapidly lead to an increase in their numbers, a process crucial for righting infection. The active form of the protein is found extracellularly as a homodimer. Non-limiting examples include UniProt entries P04141, Q647J8, Q2VPI8, and homologues thereof.

Colony stimulating factor 3 (granulocyte) (synonyms: filgrastim, GCSF, G-CSF, granulocyte colony-stimulating factor precursor, lenograstim, MGC45931, pluripoietin) is a growth factor or cytokine produced by a number of different tissues to stimulate the bone marrow to produce granulocytes and stem cells. G-CSF then stimulates the bone marrow to pulse them out of the marrow into the blood. It also stimulates the survival, proliferation, differentiation, and function of neutrophil precursors and mature neutrophils. Non-limiting examples include UniProt entries P09919, Q8N4W3, Q6FH65, and homologues thereof.

Gremlin 1, cysteine knot superfamily, homolog (Xenopus laevis) (synonyms: CKTSFIB1, Cysteine knot superfamily 1, BMP antagonist 1, DAND2, down-regulated in Mos-transformed cells protein, DRM, gremlin, GREMLIN, gremlin-1 precursor, 1110-2, increased in high glucose protein 2, MGC126660, PIG2, proliferation-inducing gene 2 protein) is a member of the BMP (bone morphogenic protein) antagonist family. Like BMPs, BMP antagonists contain cystine knots and typically form homo- and heterodimers. The antagonistic effect of the secreted glycosylated protein is likely due to its direct binding to BMP proteins. Non-limiting examples include UniProt entries 060565, Q52LV3, Q8N936, and homologues thereof.

Gremlin 2, cysteine knot superfamily, homolog (Xenopus laevis) (synonyms: CKTSF1B2, Cysteine knot superfamily 1, BMP antagonist 2, DAND3, FLJ21195, gremlin-2 precursor, Prdc, PRDC, protein related to DAN and cerberus) is a cytokine that inhibits the activity of BMP2 and BMP4 in a dose-dependent manner. It antagonizes BMP4-induced suppression of progesterone production in granulosa cells. Non-limiting examples include UniProt entries Q9H772 and Q86UD9, and homologues thereof.

Heparin-binding EGF-like growth factor (synonyms: Diphtheria toxin receptor, DTR, DT-R, DTS, HB-EGF, HEGFL, heparin-binding EGF-like growth factor precursor) is a member of the EGF family of proteins. It has been shown to play a role in wound healing, cardiac hypertrophy and heart development and function. Non-limiting examples include UniProt entries Q99075, Q9UMJ6, Q53H93, and homologues thereof.

Natriuretic peptide precursor B (synonym: brain natriuretic peptide, B-type natriuretic peptide, GC-B) is a 32 amino acid polypeptide secreted by the ventricles of the heart in response to excessive stretching of myocytes (heart muscles cells) in the ventricles. At the time of release, a co-secreted 76 amino acid N-terminal fragment (NT-proBNP) is also released with BNP. BNP binds to and activates NPRA in a similar fashion to atrial natriuretic peptide (ANP) but with 10-fold lower affinity. The biological half-life of BNP, however, is twice as long as that of ANP. Both ANP and BNP have limited ability to bind and activate NPRB. Physiologic actions of BNP and ANP include decrease in systemic vascular resistance and central venous pressure as well as an increase in natriuresis. Thus, the resulting effect of these peptides is a decrease in cardiac output and a decrease in blood volume. Non-limiting examples include UniProt entries P16860, Q6FGY0, Q9P2Q7, and homologues thereof.

Pleiotrophin (synonyms: HARP, HBBM, HB-GAM, HBGF8, HBGF-8, HBNF, HBNF1, HBNF-1, heparin-binding brain mitogen, heparin-binding growth-associated molecule, heparin-binding growth factor 8, heparin-binding neurite outgrowth-promoting factor 1, NEGF1, OSF-1, osteoblast-specific factor 1, pleiotrophin precursor) is an 18-kDa growth factor that has a high affinity for heparin. Pleiotrophin was initially recognized as a neurite outgrowth-promoting factor present in rat brain around birth and as a mitogen toward fibroblasts isolated from bovine uterus tissue. Non-limiting examples include UniProt entries P21246, Q5U0B0, Q6ICQ5, and homologues thereof.

Pre-B-cell colony enhancing factor 1 (synonyms: MGC117256, NAmPRTase, Nampt, NAMPT, nicotinamide phosphoribosyltransferase, PBEF, pre-B-cell colony-enhancing factor 1, pre-B cell-enhancing factor, visfatin) is a nicotinamide phosphoribosyltransferase (Nampt) enzyme that catalyzes first step in the biosynthesis of NAD from nicotinamide. This protein has also been reported to be a cytokine (PBEF) that promotes B cell maturation and inhibits neutrophil apoptosis. It enhances the maturation of B cell precursors in the presence of Interleukin (IL)-7 and stem cell factor. Non-limiting examples include UniProt entries P43490, Q3KQV0, Q8WW95, and homologues thereof.

Tumor necrosis factor (ligand) superfamily, member 4 (tax-transcriptionally activated glycoprotein 1, 34 kDa) (synonyms: CD134L, CD252, CD252 antigen, Glycoprotein Gp34, gp34, GP34, OX40L, OX-40L, OX40 ligand, OX40L, TAX transcriptionally-activated glycoprotein 1, tumor necrosis factor ligand superfamily member 4, TXGP1) is a cytokine that belongs to the tumor necrosis factor (TNF) ligand family. This cytokine is a ligand for receptor TNFRSF4/OX4. It is found to be involved in T cell antigen-presenting cell (APC) interactions. In surface Ig- and CD40-stimulated B cells, this cytokine along with CD70 has been shown to provide CD28-independent costimulatory signals to T cells. This protein and its receptor are reported to directly mediate adhesion of activated T cells to vascular endothelial cells. Non-limiting examples include UniProt entries P23510, Q8IV74, Q5JZA5, and homologues thereof.

Tumor necrosis factor receptor superfamily, member 11b (synonyms: MGC29565, OCIF, OPG, osteoclastogenesis inhibitory factor, osteoprotegerin, TR1, tumor necrosis factor receptor superfamily member 11B precursor) is a cytokine and a member of the tumor necrosis factor (TNF) receptor superfamily. It inhibits the differentiation of macrophages into osteoclasts and also regulates the resorption of osteoclasts in vitro and in vivo. Osteoprotegerin is a RANK homolog, and works by binding to RANK ligand on osteoblast/stromal cells, thus blocking the RANK-RANK ligand interaction between osteoblast/stromal cells and osteoclast precursors. This has the effect of inhibiting the differentiation of the osteoclast precursor into a mature osteoclast. Non-limiting examples include UniProt entries O00300, Q53FX6, O60236, and homologues thereof.

In further embodiments, certain other combinations of components identified to be present within secreted products from cultures of embryonic germ (EG) cell derivatives or umbilical cord blood stem cells are found to provide beneficial properties for wound treatment, such as but not limited to increasing type V collagen production in the skin, increase vascularization of the skin; or increase glandular secretions in the epidermal layer of the skin. Any one or two, or all three, of the aforementioned activities are embraced in other embodiments.

Such compositions comprise, in one embodiment, elastase 2A; prostaglandin I2; prostaglandin E2; amphiregulin; fibroblast growth factor 2; fibroblast growth factor 7; G protein-coupled receptor, family C, group 5, member B; and GABA(a) receptor-associated protein like 1.

Compositions for treatment or prevention of the various conditions described herein may comprise the individual components at a concentration from about 0.000001 to 1.0%. In other embodiments, the components are present in the same relative proportions as they are present in conditioned medium. In a further embodiment the components are present at the same relative proportions as they are present in conditioned medium, but at an overall concentration higher than or lower than present in conditioned medium. As noted herein below, in certain formulations such as a liposome formulation, the aforementioned components may be present at higher amounts within the interior of the liposome but at a lower concentration in the formulation based on the overall liposome content of the formulation.

The aforementioned components of the compositions embodied herein are described in further detail below. The descriptions that are provided are meant to be merely exemplary of the components and biological activities, are, not intending to be limiting. A non-limiting list of synonyms and a brief description of the individual components is provided yet Applicant is not bound to the descriptions thereof. Reference is provided to the Uniprot (Universal Protein Resource) data base (www.pir.uniprot.org), a source of sequence and other information on these components. Exemplary, non-limiting information is provided. Homologues of the components include, in the instance where components are proteins, of homologues of various mammalian species, including but not limited to human, chimpanzee, pig, rat, and mouse, as well as muteins and other functional variants, analogs, and modifications of the proteins that provide the same or similar biological activity. Homologues of the organic compounds herein include analogs, variants, adducts, modifications and the like, with the same or similar biological activity as the compound described. Analogs may have enhanced activity.

Elastase 2A, prostaglandin I2 and prostaglandin E2 are described above.

Amphiregulin (synonyms: amphiregulin precursor, AR, schwannoma-derived growth factor, Sdgf, SDGF) is a member of the EGF family of proteins and is a major autocrine growth factor for cultured human keratinocytes and probably plays a role in the aberrant keratinocyte growth of hyperproliferative disorders. Non-limiting examples include UniProt entry P24338 and homologues thereof.

Fibroblast growth factor 2 (synonyms: basic fibroblast growth factor, FGF2, BFGF, FGFB, HBGF-2, HBGH-2, heparin-binding growth factor 2 precursor, prostatropin) is a member of the fibroblast growth factor family. In normal tissue, basic fibroblast growth factor is present in basement membranes and in the subendothelial extracellular matrix of blood vessels. It stays membrane-bound as long as there is no signal peptide. Non-limiting examples include UniProt entries P09038, Q9UC54, and Q7KZ72, and homologues thereof.

Fibroblast growth factor 7 (synonyms: FGF-7, HBGF-7, keratinocyte growth factor precursor, KGF) is a member of the fibroblast growth factor family that stimulates the growth of epithelial cells, but lacks mitogenic activity on fibroblasts or endothelial cells. FGF7 is a single polypeptide chain of about 28 kD that has predominant activity in keratinocytes. Non-limiting examples include UniProt entries P21781, Q6RK68, Q6FGV5, and homologues thereof.

G protein-coupled receptor, family c, group 5, member b (synonyms: GPRC5B, O-protein coupled receptor family C group 5 member 13 precursor, RAIG2, RAIG-2, retinoic acid-induced gene 2 protein) is characterized by a signature 7-transmembrane domain motif. The specific function of this protein is unknown; however, this protein may mediate the cellular effects of retinoic acid on the G protein signal transduction cascade. The protein encoded by this gene is a member of the type 3 G protein-coupled receptor family. Non-limiting examples include UniProt entries Q9NZH0, O75205, Q8NBZ8, and homologues thereof.

GABA(a) receptor-associated protein like 1 (synonyms: APG8L, ATG8, early estrogen-regulated protein, GABA(A) receptor-associated protein-like 1, gamma-aminobutyric acid receptor-associated protein-like 1, gec1, GEC1, GEC-1, glandular epithelial cell protein 1) was described by Vemier-Magnin et al., 2001, A novel early estrogen-regulated gene gec1 encodes a protein related to GABARAP. Biochem Biophys Res Commun. 284:118-25. Non-limiting examples include UniProt entries Q9HOR8 and Q6FIE6, and homologues thereof.

To prepare a composition comprising any of the compositions described previously, the individual components may be purchased from commercial suppliers that sell proteins and other biochemicals, or expressed from constructs prepared from purchased cDNA or identified from a cDNA library. In other embodiments, the proteins can be isolated from embryonic germ cell derivatives conditioned medium or other conditioned medium from cells that produce the desired component, or purified from other biological sources. Other means of procuring or producing the components are well known to the skilled artisan. Companies that sell biochemicals, proteins, and cDNAs include Sigma Life Sciences, Invitrogen, OriGene Technologies, which are merely a few examples of numerous commercial sources.

In further embodiments, any of the foregoing compositions can optionally also include the following components: MgSO₄ (anhydrous); CaCl₂ (anhydrous); KCl; NaCl; NaHCO₃; NaH₂PO₄.H₂O; L-alanine; L-arginine.HCl; L-asparagine.H₂O; L-aspartic acid; L-cysteine.HCl.H₂O; L-cystine.2HCl; L-glutamic acid; L-glutamine; glycine; L-histidine.HCl.H₂O; L-isoleucine; L-leucine; L-lysine.HCl; L-methionine; L-phenylalanine; L-proline; L-serine; L-threonine; L-tryptophan; L-tyrosine.2Na.2H₂O; L-valine; ascorbic acid; biotin; D-calcium pantothenate; i-inositol; nicotinamide; pyridoxine.HCl; riboflavin; thiamine.HCl; vitamin B12; choline chloride; folic acid; D-glucose; lipoic acid; sodium pyruvate; thymidine; adenosine; cytidine; guanosine; uridine; 2′-deoxyadenosine; 2′-deoxycytidine.HCl; 2′-deoxyguanosine; and fetal calf serum. In other embodiments, the concentrations of the components are as follows: MgSO₄ (anhydrous; 97.67 mg/L); CaCl2 (anhydrous; 200 mg/L); KCl (400 mg/L); NaCl (6800 mg/L); NaHCO₃ (2200 mg/L); NaH2PO₄.H2O (140 mg/L); L-alanine (25 mg/L); L-arginine.HCl (105 mg/L); L-asparagine.H₂O (50 mg/L); L-aspartic acid (30 mg/L); L-cysteine.HCl.H₂O (100 mg/L); L-cystine.2HCl (31 mg/L); L-glutamic acid (75 mg/L); L-glutamine (292 mg/L); glycine (50 mg/L); L-histidine.HCl.H2O (31 mg/L); L-isoleucine (52.4 mg/L); L-leucine (52 mg/L); L-lysine.HCl (73 mg/L); L-methionine (15 mg/L); L-phenylalanine (32 mg/L); L-proline (40 mg/L); L-serine (25 mg/L); L-threonine (48 mg/L); L-tryptophan (10 mg/L); L-tyrosine.2Na.2H2O (52 mg/L); L-valine (46 mg/L); ascorbic acid (50 mg/L); biotin (0.1 mg/L); D-calcium pantothenate (1 mg/L); i-inositol (2 mg/L); nicotinamide (1 mg/L); pyridoxine.HCl (1 mg/L); riboflavin (0.1 mg/L); thiamine.HCl (1 mg/L); vitamin B12 (1.36 mg/L); choline chloride (1 mg/L); folic acid (1 mg/L); D-glucose (1000 mg/L); lipoic acid (0.2 mg/L); sodium pyruvate (110 mg/L); thymidine (10 mg/L); adenosine (10 mg/L); cytidine (10 mg/L); guanosine (10 mg/L); uridine (10 mg/L); 2′-deoxyadenosine (10 mg/L); 2′-deoxycytidine.HCl (11 mg/L); 2′-deoxyguanosine (10 mg/L); and fetal calf serum (2% v/v). In a further embodiment the aforementioned composition can also contain 1% of a 100× solution of glutamine-penicillin-streptomycin (where the 100× solution contains, in one milliliter 10 mM citrate buffer, 10,000 units of penicillin base [as penicillin G, sodium salt], 10 mg of streptomycin base [as streptomycin sulfate], and 29.2 mg of L-glutamine). The foregoing concentrations may be proportionally diluted or concentrated in other embodiments herein. In certain embodiments, the aforementioned culture medium components are present at a concentration from that used to grow embryonic germ cells or umbilical cord blood stem cell to about 100-fold less. In one embodiment, for example, fetal calf serum is present at about 2%, the same concentration present in culture medium, or as low as 0.02% (v/v).

In one embodiment, the aforementioned compositions are provided in a liposome or microencapsulated formulation contained within a carrier, for application to the skin or parenterally. Liposomal formulations are well known in the art, including those for topical and parenteral formulations. Liposome delivery has been utilized as a pharmaceutical delivery system for many for a variety of applications [see Langer, Science, 1990, 249:1527-1533; Treat et al., in Liposomes in the Therapy of Infectious Disease and Cancer, Lopez-Berestein and Fidler (eds.), Liss: New York, pp. 353-365 (1989); Lopez-Berenstein, ibid., pp. 317-327]. A general discussion of liposomes and liposome technology can be found in a three volume work entitled “Liposome Technology” edited by G. Gregoriadis, 1993, published by CRC Press, Boca Raton, Fla. In one non-limiting embodiment, a liposome composed of phosphatidyl choline, phosphatidyl ethanolamine, oleic acid and cholesteryl hemisuccinate is used. The liposome-encapsulated composition can also be combined with an aesthetically or pharmaceutically-acceptable base for topical application or parenteral administration, as described below.

In the preparation of a wound healing composition for the uses herein, the growth medium from EG cell derivatives, stem cells, or any other cell embodied herein can be used directly, or it may be concentrated, purified, lyophilized, or fractionated, by way of example. In one embodiment, lyophilized condition medium from EG cell derivatives is provided in a wound healing composition at a concentration of about 0.001% to about 10%. In another embodiment, conditioned medium from EG cell derivatives is formulated directly into a wound healing formulation, where the conditioned medium comprises from about 1% to about 90% of the composition. As will be noted in other embodiments, fractionation of conditioned medium can be performed and components therein provided in a wound healing composition. Individual components as described in the other embodiments above can each be present from about 0.000001 to 1%. These are merely illustrative of the formulation and are not meant to be limiting.

The secreted products or particular components derived therefrom or based thereon can be provided in a composition comprising at least one carrier suitable for treatment of wounds. Depending on the type and location of the wound, and whether the composition of the invention can be implanted surgically, or injected or not, will govern the appropriate selection of carrier and form of the composition. In the embodiments wherein the composition in liquid, semi-solid or solid form can be applied to a wound, suitable carriers include such components as emollients, emulsifiers, diluents, preservatives, solubilizers and/or carriers. In another embodiment where the composition of the invention is provided in a delivery vehicle that can be placed on the wound, in one typical embodiment, a hydrogel carrier or dressing is used. In another embodiment, a biodegradable polymer or other composition comprising the secreted products is implanted or provided in a surgical site; release of the secreted products from the composition occurs and the composition biodegrades. In other embodiments, in particular for a superficial wound or one that is at least accessible from the surface of the body, the formulation can be applied as a liquid or gel to the skin and optionally rubbed on or in by the user, or spread on the skin and occluded by a bandage or other device to maintain contact for a period of time. In other embodiments, a semisolid hydrogel formulation comprising secreted products is placed on the wound, and allowed to remain in place as secreted products interact therewith. It may be covered with an occlusive bandage or other device to maintain moisture. After a sufficient period of time, the hydrogel material is removed and discarded.

In one embodiment, secreted products from LVEC cells are used in the aforementioned formulations. In another embodiment, secreted products from SDEC cells are used in the aforementioned formulations. In yet another embodiment, secreted products from embryoid body derived cells are used in the aforementioned formulations. In still a further embodiment, secreted products from embryonic germ cells derivatives are used in the aforementioned formulations. In yet still a further embodiment, secreted products from embryonic stem cells are used in the aforementioned formulations. In a still further embodiments are the compositions comprising the protein and/or organic molecule components identified in condition media shown to have enhanced wound healing properties. In another embodiment, a composition comprising elastase 2A; prostaglandin I2; prostaglandin E2; adam metallopeptidase with thrombospondin type 1 motif 5; bone morphogenetic protein 1; bone morphogenetic protein 6; chemokine (C-C motif) ligand 2; chemokine (C-C motif) ligand 20; chemokine (C-X-C motif) ligand 1; chemokine (C-X-C motif) ligand 2; chemokine (C-X-C motif) ligand 3; chemokine (C-X-C motif) ligand 5; chemokine (C-X-C motif) ligand 6; chemokine (C-X-C motif) ligand 9; colony stimulating factor 2; colony stimulating factor 3; gremlin 1, cysteine knot superfamily, homolog (Xenopus laevis); gremlin 2, cysteine knot superfamily, homolog (Xenopus laevis); heparin-binding EGF-like growth factor; natriuretic peptide precursor B; pleiotrophin; pre-B-cell colony enhancing factor 1; tumor necrosis factor (ligand) superfamily, member 4; and tumor necrosis factor receptor superfamily, member 11b is provided. In another embodiment, a composition comprising elastase 2A; prostaglandin I2; prostaglandin E2; amphiregulin; fibroblast growth factor 2; fibroblast growth factor 7; G protein-coupled receptor, family C, group 5, member B; and GABA(a) receptor-associated protein like 1 is provided. Individual components can each be present from about 0.000001 to 1%.

The compositions herein may contain a wide range of additional, optional components. Formulations for use on the skin can comprise components commonly used in the cosmetics field. Examples include: absorbents, abrasives, anticaking agents, antifoaming agents, antioxidants, binders, biological additives, buffering agents, bulking agents, chelating agents, chemical additives, colorants, cosmetic biocides, denaturants, external analgesics, film formers, fragrance components, humectants, opacifying agents, pH adjusters, plasticizers, preservatives, propellants, reducing agents, skin-conditioning agents (emollient, humectants, miscellaneous, and occlusive), skin protectants, solvents, foam boosters, hydrotropes, solubilizing agents, suspending agents (nonsurfactant), sunscreen agents, ultraviolet light absorbers, waterproofing agents, and viscosity increasing agents (aqueous and nonaqueous).

Water is employed in amounts effective to form an emulsion. It is generally desirable to use water which has been purified by processes such as deionization or reverse osmosis, to improve the batch-to-batch formulation inconsistencies which can be caused by dissolved solids in the water supply. The amount of water in the emulsion or composition can range from about 15 percent to 95 weight percent, usually from about 45 to 75 percent, and typically from about 60 percent to about 75 percent.

Typical suitable emollients include mineral oil having a viscosity in the range of 50 to 500 centipoise (cps), lanolin oil, coconut oil, cocoa butter, olive oil, almond oil, macadamia nut oil, aloe extracts such as aloe vera lipoquinone, synthetic jojoba oils, natural Sonora jojoba oils, safflower oil, corn oil, liquid lanolin, cottonseed oil and peanut oil. In one embodiment, the emollient is a cocoglyceride, which is a mixture of mono, di and triglycerides of cocoa oil, sold under the trade name of Myritol 331 from Henkel KGaA, or Dicaprylyl Ether available under the trade name Cetiol OE from Henkel KGaA or a C₁₂₋₁₃ alkyl benzoate sold under the trade name Finsolv TN from Finetex. One or more emollients may be present ranging in amounts from about 1 percent to about 10 percent by weight, typically about 5 percent by weight. Another suitable emollient is DC 200 Fluid 350, a silicone fluid, available Dow Corning Corp.

Other suitable emollients include squalane, castor oil, polybutene, sweet almond oil, avocado oil, calophyllum oil, ricin oil, vitamin E acetate, olive oil, silicone oils such as dimethylopolysiloxane and cyclomethicone, linolenic alcohol, oleyl alcohol, the oil of cereal germs such as the oil of wheat germ, isopropyl palmitate, octyl palmitate, isopropyl myristate, hexadecyl stearate, butyl stearate, decyl oleate, acetyl glycerides, the octanoates and benzoates of C₁₂₋₁₅ alcohols, the octanoates and decanoates of alcohols and polyalcohols such as those of glycol and glyceryl, ricinoleates esters such as isopropyl adipate, hexyl laurate and octyl dodecanoate, dicaprylyl maleate, hydrogenated vegetable oil, phenyltrimethicone, jojoba oil and aloe vera extract.

Other suitable emollients which are solids or semi-solids at ambient temperatures may be used. Such solid or semi-solid cosmetic emollients include glyceryl dilaurate, hydrogenated lanolin, hydroxylated lanolin, acetylated lanolin, petrolatum, isopropyl lanolate, butyl myristate, cetyl myristate, myristyl myristate, myristyl lactate, cetyl alcohol, isostearyl alcohol and isocetyl lanolate. One or more emollients can optionally be included in the formulation.

A humectant is a moistening agent that promotes retention of water due to its hygroscopic properties. Suitable humectants include glycerin, polymeric glycols such as polyethylene glycol and polypropylene glycol, mannitol and sorbitol. Typically, the humectant is sorbitol, 70% USP or polyethylene glycol 400, NF. One or more humectants can optionally be included in the formulation in amounts from about 1 percent to about 10 percent by weight, typically about 5 percent by weight.

A hydrogel composition can also be used, and can include a biocompatible polymer component. The biocompatible polymer component can include one or more natural polymers, synthetic polymers, or combinations thereof. For example, the biocompatible polymer can be a polyalkylene oxide such as polyethylene glycol (PEG) or polypropylene glycol, or a derivative of PEG including but not limited to carbonates of polyethylene glycol. The hydrogel can be non-ionic, cationic or anionic. Many other hydrogel-forming polymers are known to the skilled practitioner, including those employing monomeric saccharides, amino acids, and others, to name only an exemplary few. Furthermore, various physicochemical properties are known for hydrogels, such as liquids, pastes, and membranes that can be applied to skin, for example. Various other non-limiting examples are described in US Patent Application 2005/0112151.

It may be advantageous to incorporate additional thickening agents, such as, for instance, Carbopol Ultrez, or alternatively, Carbopol ETD 2001, available from the B.F. Goodrich Co. The selection of additional thickening agents is well within the skill of one in the art.

A waterproofing or water resistance agent is a hydrophobic material that imparts film forming and waterproofing characteristics to an emulsion. A suitable waterproofing agent is a copolymer of vinyl pyrollidone and eicosene and dodecane monomers such as Ganex V 220 and Ganex V 216 Polymers, respectively, trade names of ISP Inc. of Wayne, N.J. U.S.A. Still other suitable waterproofing agents include polyurethane polymer, such as Performa V 825 available from New Phase Technologies and polyanhydride resin No. 18 available under the trade name PA-18 from Chevron.

An antimicrobial preservative is a substance or preparation which destroys, or prevents or inhibits the proliferation of, microorganisms in the composition, and which may also offer protection from oxidation. Preservatives are frequently used to make self-sterilizing, aqueous based products such as emulsions. This is done to prevent the development of microorganisms that may be in the product from growing during manufacturing and distribution of the product and during use by consumers, who may further inadvertently contaminate the products during normal use. Typical preservatives include the lower alkyl esters of para-hydroxybenzoates (parabens), especially methylparaben, propylparaben, isobutylparaben and mixtures thereof, benzyl alcohol, phenyl ethyl alcohol and benzoic acid. One preservative is available under the trade name of Germaben II from Sutton. One or more antimicrobial preservatives can optionally be included in an amount ranging from about 0.001 to about 10 weight percent, typically about 0.05 to about 1 percent.

An antioxidant is a natural or synthetic substance added to the formulation to protect from or delay its deterioration due to the action of oxygen in the air (oxidation). Anti-oxidants prevent oxidative deterioration which may lead to the generation of rancidity and nonenyzymatic browning reaction products. Typical suitable antioxidants include propyl, octyl and dodecyl esters of gallic acid, butylated hydroxyanisole (BHA, usually purchased as a mixture of ortho and meta isomers), butylated hydroxytoluene (BHT), nordihydroguaiaretic acid, Vitamin A, Vitamin E and Vitamin C. One or more antioxidants can optionally be included in the composition in an amount ranging from about 0.001 to about 5 weight percent, typically about 0.01 to about 0.5 percent.

Chelating agents are substances used to chelate or bind metallic ions, such as with a heterocyclic ring structure so that the ion is held by chemical bonds from each of the participating rings. Suitable chelating agents include ethylene diaminetetraacetic acid (EDTA), EDTA disodium, calcium disodium edetate, EDTA trisodium, EDTA tetrasodium and EDTA dipotassium. One or more chelating agents can optionally be included in the formulation in amounts ranging from about 0.001 to about 0.2 weight percent typically about 0.01% weight percent.

A pH modifier is a compound that will adjust the pH of a formulation to a lower, e.g., more acidic pH value, or to a higher, e.g., more basic pH value. The selection of a suitable pH modifier is well within the ordinary skill of one in the art.

Suitable emulsifiers for one aspect of the embodiments herein are those known in the art for producing oil-in-water and/or water-in-oil type emulsions. An aqueous external phase is preferred by many people for skin contact, since it is not as likely to produce an oily or greasy sensation when it is being applied, as is an emulsion having an oil external phase. The typical oil-in-water emulsifier has a hydrophilic-lipophilic balance (frequently abbreviated as “HLB”) value greater than about 9, as is well known in the art; however, this “rule” is known to have numerous exceptions. The chosen emulsifier, depending upon its chemical nature, will be a component of either the oil or aqueous phase, and assists with both the formation and the maintenance, or stability, of the emulsion. Suitable emulsifiers for another embodiment herein are those known in the art for producing water-in-oil type emulsions. The typical water-in-oil emulsifier has a HLB value of about 4 to about 6, as is well known in the art; however, this “rule” is also known to have numerous exceptions. Selection of suitable water-in-oil emulsifiers is well known in the formulation art.

Most of the widely used emulsifier systems for formulations can be used in the formulations herein. In some embodiment, emulsifiers are PEG-8 Distearate available under the trade name of Emerest 2712 from Henkel, PEG-5 Glyceryl Stearate available under the trade name POEM-S-105 from Riken Vitamin Oil, PEG-6 Hydrogenated Castor Oil, available under the trade name Sabowax ELI-16 from Sabo, PEG-6 Oleate, available under the trade name STEPAN PEG-300 MO from Stepan, Sorbitan Sesquioleate, available under the trade name Arlacel 83 and Arlacel C from ICI Surfactants, TEA-Stearate, available under the trade name of Cetasal from Gaftefosse S.A. Another typical emulsifier is neutralized cetyl phosphate, available under the trade name Amphisol A from LaRoche. In another embodiment the emulsifier is an Acrylate/C₁₀₋₃₀ alkyl acrylate cross polymer of C₁₀₋₃₀ alkyl acrylates and one or more monomers of acrylic acid, methacrylic acid or one of their simple esters crosslinked within allyl ether of sucrose or an allyl ether of pentaerythritol, available under the trade names of Pemulen TR from B.F. Goodrich. The amount of emulsifier used herein may present in an amount of about 0.1 to about 10% by weight, typically about 0.5 percent to about 5 percent by weight, most typically about 2 percent to about 4 percent by weight. The choice of an emulsifier is well within ordinary skill in the art and is not a critical aspect herein. Additional emulsifiers that may be employed include sorbitan triisostearate available under the trade name Crill 6 from Croda Oleochemicals, and polyglyceryl-3 distearate available under the trade name Cremophor GS 32 from BASF.

As is known in the art, the individual emulsion droplets typically have a small and uniform size because these properties result in a more stable emulsion. Conversely, a broad particle size distribution indicates that the interfacial tension between the droplets has not been substantially reduced, and thus the droplets tend to coalesce and form agglomerations that result in an unstable emulsion.

Hydrogels, further to the description above, may comprise poly(N-vinyl lactam), including homopolymers, copolymers and terpolymers of N-vinyl lactams such as N-vinylpyrrolidone, N-vinylbutyrolactam, N-vinylcaprolactam, and the like, as well as the foregoing prepared with minor amounts, for example, up to about 50 weight percent, of one of a mixture of other vinyl monomers copolymerizable with the N-vinyl lactams. Copolymers or terpolymers of poly (N-vinyl-lactam) may comprise N-vinyl-lactam monomers such as vinylpyrrolidone copolymerized with monomers containing a vinyl functional group such as acrylates, hydroxyalkylacrylates, methacrylates, acrylic acid or methacrylic acid, and acrylamides. Of the poly(N-vinyl lactam) homopolymers, the polyvinylpyrrolidone (PVP) homopolymers are preferred. Of the poly(N-vinyl lactam) copolymers, the vinyl pyrrolidone and acrylamide copolymers are typically employed. Of the poly(N-vinyl lactam) terpolymers, the vinylpyrrolidone, vinylcaprolactam, dimethylaminoethyl methacrylate terpolymers are typically used. A variety of polyvinylpyrrolidones are commercially available.

Hydrogels are stable and maintain their physical integrity after absorbing large quantities of liquid. The gels can be sterilized by radiation sterilization, autoclave or exposed to ethylene oxide. The gels are hydrophilic and capable of absorbing many times of their dry weight in water. Wetting, dispersing agents or surfactants as are known in the art may be added. Glycerin in an amount of 0 to 50 wt. %, preferably from about 5 to 40 wt. % may be added to the gel to increase tack, pliability after drying for the gel. Propylene glycol or polyethylene glycol may also be added. Other additives may be combined with the hydrogels including organic salts, inorganic salts, alcohols, amines, polymer lattices, fillers, surfactants, dyes, etc., among other components described herein.

Of course, any composition having at least secreted products from the variety of cells described herein can also include one or more other components, in addition to the carriers and excipients mentioned herein. Such at least one additional component can be another wound healing agent, that can work additively or synergistically with the secreted products embodied herein.

In certain embodiments, the formulation embodied here is in the form of an over-the-counter product. In other embodiments, the formulation is an ethical item requiring a prescription by a health care professional. In other embodiments the product is used only for patients in a hospital or other full-time medical care setting. In certain embodiments, the ethical formulation comprises a higher concentration of secreted products than the non-prescription formulation. In other embodiments, various strengths or concentrations of secreted products in a formulation are available, such that the health care professional can match a particular condition with a suitable formulation, frequency of application, duration of treatment course, for example, to optimize the desired effect for a particular patient, his or her condition, and compliance with instructions for use.

In one embodiment, the embryonic germ (EG) cell derivatives are embryoid body-derived cells. In another embodiment, the embryoid body-derived cells are LVEC cells or SDEC cells. As will be described in the examples below, EBD cultures are named such that the first two letters refer to the EG culture from which it was derived, the third letter indicates the growth media in which it was derived and is maintained and the fourth letter indicates the matrix on which it is grown.

In other embodiments, the embryonic germ (EG) derivatives are from mouse, pig, chicken, or human.

A controlled release delivery system or device can also be used, to deliver the secreted products over time. Application can be performed at a frequency specified on the package, or in accordance with a provider's recommendation or prescription. The amount of secreted products present in the formulation will depend on the potency, frequency of application, nature of the carrier, solubility, and other factors readily determinable based on the desired outcome and selection of carrier. Typically, secreted products can be present in a formulation between 0.0001% and 10% (w/v).

The following examples are intended to illustrate but not limit the invention. While they are typical of those that might be used, other procedures known to those skilled in the art may alternatively be used.

### EXAMPLES

### Example 1

### Derivation of Embryoid Germ Cell Derivatives

Human pluripotent germ cell cultures were derived from primordial germ cells, isolated and cultured as described above and in Shamblott et al., Proc. Natl. Acad. Sci. USA 95:13726-13731, 1998). Four genetically distinct human EG cell cultures were selected to represent the range of developmental stages at which human EG cultures can be initiated, with karyotypes as noted LV (46, XX), SL (46, XY), LU2 (46, XY) and SD (46, XX). These cultures were derived and cultured from 5, 6, 7, and 11 week post-fertilization primordial germ cells (PGCs), respectively. Embryoid bodies (EBs) were formed in the presence of leukemia inhibitory factor (LIF, 1000 U/ml), basic fibroblast growth factor (bFGF, 2 mg/ml), forskolin (10 μM) and 15% fetal calf serum (FCS, Hyclone). During routine growth, 1 to 5% of the multicellular EG colonies formed large fluid-filled cystic EBs that were loosely attached to a remaining EG colony or to the fibroblast feeder layer. Approximately 10 cystic EBs from each culture were dissociated by digestion 1 mg/ml in Collagenase/Dispase (Roche Molecular Biochemicals) for 30 min. to 1 hour at 37 C. Cells were then spun at 1000 rpm for 5 min.

EB constituent cells were then resuspended and replated in growth media and human extracellular matrix (Collaborative Biomedical, 5 μg/cm2), and tissue culture plastic. Cells were cultured at 37 C, 5% CO₂, 95% humidity and routinely passaged 1:10 to 1:40 by using 0.025% trypsin, 0.01% EDTA (Clonetics) for 5 min. at 37 C. Low serum cultures were treated with trypsin inhibitor (Clonetics) and then spun down and resuspended in growth media. Cell were cryopreserved in the presence of 50% FCS, 10% dimethylsulfoxide (DMSO) in a controlled rate freezing vessel, and stored in liquid nitrogen. Exemplary cell culture designations LVEC and SDEC are the cells derived as mentioned above (LV, SD) grown on human extracellular matrix (EC).

### Example 2

### Derivation of Umbilical Cord Stem Cells

Frozen human umbilical cord blood mononuclear cells were received from Cambrex, thawed according to manufacturer's recommendation and placed into 3 T75 flasks with 12 ml each of USSC media (low glucose DMEM with Glutamax, Invitrogen 10567-014), 10-7 M dexamethasone (Sigma), 100 U/ml penicillin and 0.1 mg/ml streptomycin. Media was changed after 48 hrs then every 2-3 days following. On day 14, proliferating cells were passaged 1:3 into new flasks by using 0.25% trypsin/EDTA and neutralized by trypsin neutralization solution. Every 5 to 7 days cells were similarly passaged. Aliquots of these umbilical cord stem cells (herein abbreviated “UCSC”) were cryopreserved at several passages.

The phenotype of passage 5 UCSC was CD31 (2%), CD34 (0%), CD44 (97%), CD50 (0%), CD71 (47%), CD90, (96%), CD106 (0%).

Passage 6 UCSC were plated onto a collagen I+Gelatin coated 6 well plate. Each well contained between 0.2 to 1 million cells. Cells were irradiated at 3500 RAD. HuES-2 (Harvard line, Passage 27) cells were plated into the coated wells and the media was changed to HuES media (Cowan, C. A. et. al, 2004). Typical hES colonies were observed at all UCSC densities, so 0.25 million cells per well was chosen for all future work. hES cells were passaged every 3-5 days using 0.05 trypsin/EDTA.

### Example 3

### Embryonic Stem Cells

HuES-2 were observed growing on Matrigel in the presence of UCSC conditioned media. This conditioned media was prepared by plating UCSC at 1 million cells per 10 cm plate into 12 mls of HuES media. After 24 hrs media was harvested and sterile filtered. Prior to use on huES-2 cells, 8 ng/ml FGF2 was added to the conditioned media.

### Example 4

### Secreted Products for Wound Care Uses

Conditioned media containing secreted product from LVEC cells, UCSC and embryonic stem cells as described above is collected from cell cultures, concentrated by lyophilization and a formulation is prepared therefrom in a water-soluble emollient. In one example, the formulation comprises a hydrogel wound dressing-type polymer comprising 10% lyophilized conditioned medium. In another example secreted products from umbilical cord stem cells are provided in a similar manner. Application to a wound provides an enhanced rate of healing and improved properties of the healed wound.

### Example 5

### Components from Secreted Products

Among a large number of components identified in secreted products from embryonic germ cells and umbilical cord blood stem cells, the following components were found to provide enhanced wound healing properties based on, among other activities, the ability to induce rapid growth of skin cells and increase the number of keratinocytes in the skin. The components are elastase 2A; prostaglandin I2; and prostaglandin E2; adam metallopeptidase with thrombospondin type 1 motif 5; bone morphogenetic protein 1; bone morphogenetic protein 6; chemokine (C-C motif) ligand 2; chemokine (C-C motif) ligand 20; chemokine (C-X-C motif) ligand 1; chemokine (C-X-C motif) ligand 2; chemokine (C-X-C motif) ligand 3; chemokine (C-X-C motif) ligand 5; chemokine (C-X-C motif) ligand 6; chemokine (C-X-C motif) ligand 9; colony stimulating factor 2; colony stimulating factor 3; gremlin 1, cysteine knot superfamily, homolog (Xenopus laevis); gremlin 2, cysteine knot superfamily, homolog (Xenopus laevis); heparin-binding EGF-like growth factor; natriuretic peptide precursor B; pleiotrophin; pre-B-cell colony enhancing factor 1; tumor necrosis factor (ligand) superfamily, member 4; and tumor necrosis factor receptor superfamily, member 11b.

Also present were culture medium components, including MgSO₄ (anhydrous); CaCl₂ (anhydrous); KCl; NaCl; NaHCO₃; NaH₂PO₄.H₂O; L-alanine; L-arginine.HCl; L-asparagine.H₂O; L-aspartic acid; L-cysteine.HCl.H₂O; L-cystine-2.HCl; L-glutamic acid; L-glutamine; glycine; L-histidine.HCl.H₂O; L-isoleucine; L-leucine; L-lysine.HCl; L-methionine; L-phenylalanine; L-proline; L-serine; L-threonine; L-tryptophan; L-tyrosine.2Na.2H₂O; L-valine; ascorbic acid; biotin; D-calcium pantothenate; i-inositol; nicotinamide; pyridoxine.HCl; riboflavin; thiamine.HCl; vitamin B12; choline chloride; folic acid; D-glucose; lipoic acid; sodium pyruvate; thymidine; adenosine; cytidine; guanosine; uridine; 2′-deoxyadenosine; 2′-deoxycytidine.HCl; 2′-deoxyguanosine; and fetal calf serum.

### Example 6

### Components from Secreted Products

Among a large number of other components identified in secreted products from embryonic germ cells and umbilical cord blood stem cells, the following components were found to provide enhanced wound healing properties based upon, among other activities, the ability to increase type V collagen production in the skin, increase vascularization of the skin; or increase glandular secretions in the epidermal layer of the skin. The components are elastase 2A; prostaglandin I2; prostaglandin E2; amphiregulin; fibroblast growth factor 2; fibroblast growth factor 7; G protein-coupled receptor, family C, group 5, member B; and GABA(a) receptor-associated protein like 1.

The foregoing composition also comprised the components of EG culture medium from which the individual components were identified. Those culture medium components are described in Example 5.

While certain features have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A composition for enhancing the healing of wounds, the composition comprising secreted products from embryonic germ (EG) cell derivatives, and a pharmaceutically-acceptable carrier therefor.

2. The composition of claim 2 wherein the embryonic germ (EG) cell derivatives are embryoid body-derived cells.

3. The composition of claim 2 wherein said embryoid body-derived cells are LVEC cells or SDEC cells.

4. The composition of claim 1 wherein the embryonic germ (EG) derivatives are from mouse, pig, chicken, or human.

5. The composition of claim 1 wherein enhancement of wound healing is increasing the healing rate or strength of healed wounds.

6. The composition of claim 1 wherein the carrier comprises a liquid, cream, aerosol, lotion, or hydrogel.

7. The composition of claim 1 wherein the secreted products are present in the composition at a concentration of about 0.001% to about 10%.

8. A composition for enhancing the healing of wounds, the composition comprising secreted proteins from umbilical cord stem cells, and a pharmaceutically-acceptable carrier.

9. The composition of claim 8 wherein the umbilical cord stem cells are CD34^{pos}.

10. The composition of claim 8 wherein the umbilical cord blood stem cells are adherent, CD45^{neg}, HLA class II^{neg} stem cells.

11. The composition of claim 10 wherein said adherent, CD45^{neg}, HLA class II^{neg} stem cells are CD34^{neg}, CD106^{neg}, CD44^{pos} and CD90^{pos}.

12. The composition of claim 8 wherein enhancement of wound healing is increasing the healing rate or strength of healed wounds.

13. The composition of claim 8 wherein the carrier comprises a liquid, cream, aerosol, lotion, or hydrogel.

14. The composition of claim 8 wherein the secreted products are present in the composition at a concentration of about 0.001% to about 10%.

15. A composition for enhancing the healing of wounds, the composition comprising secreted products from embryonic stem cells, and a pharmaceutically-acceptable carrier therefor.

16. The composition of claim 15 wherein enhancement of wound healing is increasing the healing rate or strength of healed wounds.

17. The composition of claim 15 wherein the carrier comprises a liquid, cream, aerosol, lotion, or hydrogel.

18. The composition of claim 15 wherein the secreted products are present in the composition at a concentration of about 0.001% to about 10%.

19. A method for enhancing the healing of wounds comprising applying to the wound a composition comprising secreted products from embryonic germ (EG) cell derivatives, and a pharmaceutically-acceptable carrier therefor.

20. The method of claim 19 wherein the embryonic germ (EG) cell derivatives are embryoid body-derived cells.

21. The method of claim 20 wherein said embryoid body-derived cells are LVEC cells or SDEC cells.

22. The method of claim 19 wherein the embryonic germ (EG) derivatives are from mouse, pig, chicken, or human.

23. The method of claim 19 wherein enhancement of wound healing is increasing the healing rate or strength of healed wounds.

24. The method of claim 19 wherein the carrier comprises a liquid, cream, aerosol, lotion, or hydrogel.

25. A method for enhancing the healing of wounds comprising applying to the wound a composition comprising secreted proteins from umbilical cord stem cells, and a pharmaceutically-acceptable carrier.

26. The method of claim 25 wherein the cells are CD34^{pos}.

27. The method of claim 25 wherein the umbilical cord blood stem cells are adherent, CD45^{neg}, HLA class II^{neg} stem cells.

28. The method of claim 27 wherein said adherent, CD45^{neg}, HLA class II^{neg} stem cells are CD34^{neg}, CD106^{neg}, CD44^{pos} and CD90^{pos}.

29. The method of claim 25 wherein enhancement of wound healing is increasing the healing rate or strength of healed wounds.

30. The method of claim 25 wherein the carrier comprises a liquid, cream, aerosol, lotion, or hydrogel.

31. A method for enhancing the healing of wounds comprising applying to the wound a composition comprising secreted proteins from embryonic stem cells, and a pharmaceutically-acceptable carrier.

32. The method of claim 31 wherein enhancement of wound healing is increasing the healing rate or strength of healed wounds.

33. The method of claim 31 wherein the carrier comprises a liquid, cream, aerosol, lotion, or hydrogel.

34. A composition for enhancement of wound healing comprising elastase 2A; prostaglandin I2; prostaglandin E2; adam metallopeptidase with thrombospondin type 1 motif 5; bone morphogenetic protein 1; bone morphogenetic protein 6; chemokine (C-C motif) ligand 2; chemokine (C-C motif) ligand 20; chemokine (C-X-C motif) ligand 1; chemokine (C-X-C motif) ligand 2; chemokine (C-X-C motif) ligand 3; chemokine (C-X-C motif) ligand 5; chemokine (C-X-C motif) ligand 6; chemokine (C-X-C motif) ligand 9; colony stimulating factor 2; colony stimulating factor 3; gremlin 1, cysteine knot superfamily, homolog (Xenopus laevis); gremlin 2, cysteine knot superfamily, homolog (Xenopus laevis); heparin-binding EGF-like growth factor; natriuretic peptide precursor B; pleiotrophin; pre-B-cell colony enhancing factor 1; tumor necrosis factor (ligand) superfamily, member 4; and tumor necrosis factor receptor superfamily, member 11b.

35. The composition of claim 34 further comprising MgSO₄ (anhydrous); CaCl₂ (anhydrous); KCl; NaCl; NaHCO₃; NaH₂PO₄.H₂O; L-alanine; L-arginine.HCl; L-asparagine.H₂O; L-aspartic acid; L-cysteine.HCl.H₂O; L-cystine-2.HCl; L-glutamic acid; L-glutamine; glycine; L-histidine.HCl.H₂O; L-isoleucine; L-leucine; L-lysine.HCl; L-methionine; L-phenylalanine; L-proline; L-serine; L-threonine; L-tryptophan; L-tyrosine.2Na.2H₂O; L-valine; ascorbic acid; biotin; D-calcium pantothenate; i-inositol; nicotinamide; pyridoxine.HCl; riboflavin; thiamine.HCl; vitamin B12; choline chloride; folic acid; D-glucose; lipoic acid; sodium pyruvate; thymidine; adenosine; cytidine; guanosine; uridine; 2′-deoxyadenosine; 2% deoxycytidine.HCl; 2′-deoxyguanosine; and fetal calf serum.

36. A method for enhancing wound healing comprising applying to a wound a composition of claim 34 or 35.

37. A composition for enhancement of wound healing comprising elastase 2A; prostaglandin I2; prostaglandin E2; amphiregulin; fibroblast growth factor 2; fibroblast growth factor 7; G protein-coupled receptor, family C, group 5, member B; and GABA(a) receptor-associated protein like 1.

38. The composition of claim 37 further comprising MgSO₄ (anhydrous); CaCl₂ (anhydrous); KCl; NaCl; NaHCO₃; NaH₂PO₄H₂O; L-alanine; L-arginine.HCl; L-asparagine.H₂O; L-aspartic acid; L-cysteine.HCl.H₂O; L-cystine.2.HCl; L-glutamic acid; L-glutamine; glycine; L-histidine.HCl.H₂O; L-isoleucine; L-leucine; L-lysine.HCl; L-methionine; L-phenylalanine; L-proline; L-serine; L-threonine; L-tryptophan; L-tyrosine.2Na.2H₂O; L-valine; ascorbic acid; biotin; D-calcium pantothenate; i-inositol; nicotinamide; pyridoxine.HCl; riboflavin; thiamine.HCl; vitamin B12; choline chloride; folic acid; D-glucose; lipoic acid; sodium pyruvate; thymidine; adenosine; cytidine; guanosine; uridine; 2′-deoxyadenosine; 2′-deoxycytidine.HCl; 2′-deoxyguanosine; and fetal calf serum.

39. A method for enhancing wound healing comprising applying to a wound a composition of claim 37 or 38.
